(19) United States
(12) Patent Application Publication
(10) Pub. No.: US 2020/0139163 A1
(43) Pub. Date: May 7, 2020

(54) Compositions For Removing Surface Coatings

(71) Applicant: Jake Palmer, Coto de Caza, CA (US)

(72) Inventor: Jake Palmer, Coto de Caza, CA (US)

(21) Appl. No.: 16/183,589

(22) Filed: Nov. 7, 2018

(51) Int. Cl. A61Q 3/04; C09D 9/04; C09D 9/00; A61K 8/60; A61K 8/37; A61K 8/49; A61K 8/86; A61K 8/36; A61K 8/31

(52) U.S. Cl. CPC A61Q 3/04; C09D 9/04; C09D 9/005; A61K 8/604; A61K 8/31; A61K 8/4973; A61K 8/86; A61K 8/361; A61K 8/37

(57) ABSTRACT

A low VOC composition for removing coatings from a wide variety of surfaces that is biodegradable, made from renewable resources, and environmentally friendly. The composition further pertains to products which can remove a surface coating with little mechanical action.

### FIELD OF THE INVENTION

The present invention relates to reduced VOC (volatile organic carbon) and reduced toxicity compositions for removal of coatings from surfaces. The present invention is directed towards compositions of surface coatings removal products and methods of use.

### BACKGROUND OF THE INVENTION

Many of the current cleaning compositions are being removed from the market because of toxicity and environmental concerns. Methylene chloride (dichloromethane) is currently being phased out. Mixtures using aromatic solvents containing toluene, benzene and other aromatics are known to emit air pollutants and are toxic.

The current composition is comprised of chemicals of low toxicity, many of which are renewable and can be made from sugar, grains, and cellulose. The compositions are formulated with reduction in volatile organic carbon (VOCs) as an objective as well as reduced toxicity to the environment.

Conventional solvents used for removing of coatings include trichloroethylene and mineral spirits for degreasing, hydrocarbons and chlorinated hydrocarbons, for general purpose removal, and methylene chloride in paint striper. Others have addressed this issue by formulating epoxidized soy or other natural feed stock esters (U.S. Pat. No. 7,951,766) with an abrasive scrubbing pad to assist in the removal of the paint. The nail polish remover composition in U.S. Pat. No. 10,085,926 does not use protic solvents, and the compositions requires keratin and cocoa butter. The terms, non-volatile and volatile, are not related to evaporation at room temperature. Volatile and non-volatile are measured by weight by weight loss at one hour at 110° C. U.S. Pat. No. 9,138,391 uses alcohols and esters mixed with water. U.S. Pat. No. 8,835,369 utilizes glycerols.

The unsafe and toxic properties of the chemicals used in compositions for removal of coatings from surfaces limit their use. There is a need for coating removal compositions that are non-toxic, environmentally friendly and biodegradable, while having the strength to remove most coatings from various surfaces.

All publications, patent applications, patents, and other references mentioned herein are incorporated by reference in their entirety. The materials, methods and examples given are illustrative only and not intending to be limiting.

### Definitions

In accordance with the present invention and as used herein, the following items are defined with the following meanings unless explicitly stated otherwise. Unless otherwise defined, all technical and scientific terms used herein have the same meaning as commonly understood by one of ordinary skill in the art to which this invention belongs.

The term, HLB, refers to the Hydrophilic-Lipophilic Balance value of a surfactant (Adamson, A. W, Physical Chemistry of Surfaces, pg. 505-507).

The term, VOC, refers to volatile organic compounds, based on measurement by the weight loss at 110° C. for 1 hour where the percentage of water, methyl acetate, and acetone are subtracted. Methyl acetate and acetone are exempt compounds by EPA.
Low VOC, as defined herein, is 25% or lower
High VOC, as defined herein, is 50% or greater

The term, super glue, refers to ethyl cyanoacrylate compound.

The following chemical names used herein and their common or trademark names/synonyms and associated CAS Registry number:

Methyl 5-methyl-2-furorate; Methyl 5-methylfuran-2-carboxylate; RN 2527-96-0

Potassium oleate; Trenamine D-200; RN 143-18-0

Polysorbate 80; Polyoxyethylene sorbitan monooleate; Tween® 80; RN 9005-65-6

d-Limonene; 1-methyl-4-prop-1-en-2-ylcyclohex-1-ene; Dipentene; RN 138-86-3

Isopropyl alcohol; IPA; 2-propanol; propan-2-ol; RN 67-63-0

Methyl lactate; Methyl 2-hydroxypropanoate; RN 547-64-8

Methyl Acetate: Methyl ethanoate; acetic acid, methyl ester; RN 79-20-9

PEG 7 glyceryl cocoate; Polyethylene glycol (7) glyceryl monococoate; RN 68201-46-7

PVC; Polyvinyl chloride; Polychloroethylene; RN 9002-86-2

CPVC; PVC-C; Chlorinated polyvinyl chloride; RN 68648-82-8

PETE; PET; Polyethylene terephthalate; RN 25038-69-9

### SUMMARY OF INVENTION

There is a need for coating removal compositions that are non-toxic, environmentally friendly and biodegradable, while having the strength to remove most coatings from various surfaces.

The present invention is directed towards a coating removal composition that is useful for removal of coatings from surfaces. Many aspects and embodiments are described herein, and are merely exemplary, not limiting. After reading this specification those skilled in the art will appreciate that other aspects and embodiments are possible without depart from the scope of the present invention.

In some embodiments a coating removal composition can be a mixture of compounds selected from sugar derived esters, a surfactant or surfactants, a co-solvent, and optionally a scent.

In other embodiments a coating removal composition can be a mixture of compounds selected from sugar derived esters, methyl lactate, a surfactant or surfactants, a solvent, and optionally a scent.

In additional embodiments a coating removal composition can be a mixture of compounds selected from methyl acetate, methyl lactate, methyl-5-methylfuroate (Me408), d-limonene, a surfactant or surfactants, an optional solvent, and optionally a scent.

In some embodiments a coating removal composition can be a mixture of compounds selected from methyl acetate, methyl lactate, d-limonene, a surfactant or surfactants, an optional solvent, and optionally a scent. There was the surprising finding that the mixtures worked for various coatings on a variety of surfaces.

### DETAILED DESCRIPTION OF THE INVENTION

The present invention is drawn towards a composition for removing coatings from surfaces containing sugar derived esters. These esters can be derived fermentation of sugars or carbohydrates or by direct chemical conversion of sugars or carbohydrates, which are renewable resources. Sugar fermentation is well known, and a chemical synthesis is described in U. S. Pat. No. 9,108,940. These esters are easily biodegradable. The composition may optionally include a scent, surfactant, surfactants, or a co-solvent.

In additional embodiments a coating removal composition can be a mixture of compounds selected from methyl lactate, Me408, d-limonene, a surfactant system, an optional solvent, and optionally a scent.

In some configurations a coating removal composition can be at least 30% or at 35%, or at least 40% of first sugar derived ester by weight, about 5% or about 10% of second sugar derived ester by weight, about 0% to 10% by weight of methyl-5-methylfuroate (Me408), at least 4%, or at least 6%, or at least 8%, or at least 10% by weight of d-limonene, at least 5%, at least 10%, or at least 15% by weight of a surfactant with the remainder being a solvent.

In some configurations a coating removal composition can be a mixture of compounds selected from methyl acetate, methyl lactate, d-limonene, a surfactant, an optional solvent, and optionally a scent.

In various configurations a coating removal composition can be a composition with about 5% to about 20% by weight of water as a solvent.

All chemicals have an HLB which can be calculated. A surfactant is selected based on desired HLB of a solution. To assist in removing coatings HLBs of coating removal composition is based on HLB of coatings. A surfactant of the present configurations can be characterized in part by its Hydrophilic-Lipophilic Balance (HLB) value. A known property of surfactant systems is that they can be mixed to achieve a desired HLB. Those skilled in the art can obtain similar results by using different surfactants. In some configurations in coating removal composition containing esters and a surfactant, the surfactant can be any non-ionic surfactant with a Hydrophilic-Lipophilic Balance (HLB). In the various configurations, a surfactant can be medium (HLB about 10) to a high HLB (about 20). As used herein a medium HLB is an HLB from 3-13 and a high HLB is a HLB above 13.

Non-limiting examples of surfactants and their HLB values are given in Table A.

TABLE A

| Surfactant | Synonym | CAS RN | HLB |
|---|---|---|---|
| Polyoxyethylene | Polysorbate 80 | 9005-65-6 | 10 |
| sorbitan | Tween ® 80 | | |
| monooleate | | | |
| Potassium oleate | Trenamine D-200 | 143-18-0 | 20 |
| Sorbitan monooleate | SPAN ® 80 | 1338-43-8 | 4.3 |
| Sorbitan monostearate | SPAN ® 60 | 1338-41-6 | 4.7 |
| PEG-7 glyceryl | Polyethylene glycol (7) | 68201-46-7 | 10 |
| cococate | glyceryl monococoate | | |
| Sodium Oleate | | 143-19-1 | 18 |
| Sodium lauryl sulfate | Sodium dodecyl sulfate | 151-21-3 | 40 |
| Sorbitan monolaurate | SPAN ® 20 | 26266-57-9 | 6.7 |
| Polyoxyethylene | TWEEN ® 61 | 1338-39-2 | 8.6 |
| sorbitan | | | |
| monostearate | | | |
| TRITON RW-50 | Amine ethoxylate | 73138-27-9 | 12-14 |

Suitable surfactants for use in formulations of the present invention include medium HLB surfactants such as polyoxyethylene sorbitan monooleate (Polysorbate 80; MilliporeSigma (Sigma-Aldrich); St. Louis, MO; HLB 10) and high surfactants such as potassium oleate (Alfa Chemistry, Holtsville, NY; HLB 20).

### FORMULATION EXAMPLES

The following Examples illustrate certain aspects and advantages of the present invention, which should be understood not to be limited thereby.

The following tables detail examples of various formulations. The raw materials were weighed out on an analytical balance and added to a round bottom flask with a stir bar for mixing. Ingredients are listed in the order as added and mixed until the solution was uniform, and all materials were dissolved. The solution was mixed for about 30 minutes. All weights given are in grams.

TABLE 1

| | Example | Example | Example | Example | Example | Example |
|---|---|---|---|---|---|---|
| | 1 | 2 | 3 | 4 | 5 | 6 |
| Potassium | 4.0 | 4.0 | 2.0 | 4.0 | 4.0 | |
| Oleate | | | | | | |
| Polysorbate | 6.0 | 6.0 | 6.0 | 6.0 | 6.0 | |
| 80 | | | | | | |
| d-Limonene | 10.1 | 5.0 | 2.5 | 10.1 | 10.1 | 2.01 |
| Isopropyl | 14.2 | 14.2 | 14.2 | 2.0 | 2.0 | |
| Alcohol | | | | | | |
| Methyl | 13.1 | 13.1 | 13.1 | 13.1 | 13.1 | 18.0 |
| Lactate | | | | | | |
| Methyl | | 5.1 | 9.6 | 12.2 | 12.2 | |
| Acetate | | | | | | |
| Water | 8.0 | 8.0 | 8.0 | 8.0 | 8.0 | |

TABLE 2

| | | | Example 9 | Example 10 | Example 11 | Example 12 |
|---|---|---|---|---|---|---|
| | Example | Example | D1 Low | D2 Low | D3 Low | D4 Low |
| | 7 | 8 | VOC | VOC | VOC | VOC |
| Potassium | 2.0 | 2.0 | 4.0 | 4.0 | 4.0 | 4.0 |
| Oleate | | | | | | |
| Polysorbate 80 | | | 6.0 | 6.0 | 10.0 | 6.0 |
| PEG 7 glyceryl | 2.0 | 2.0 | | | | |
| cocoate | | | | | | |
| d-Limonene | 2.0 | 2.0 | 5.1 | 5.1 | 5.1 | 5.1 |
| Acetone | 7.1 | 15.0 | | | | |
| Methyl Lactate | 18.0 | 9.0 | 5.1 | 5.1 | 5.1 | 5.1 |
| Water | | | 8.0 | 8.0 | | |
| Methyl Acetate | | 9.0 | 20.2 | 27.3 | 31.3 | 20.2 |
| VOC | | | 17.08% | 20.46% | 10.45% | 15.47% |

TABLE 3

| | Example 13 | Example 14 | | | |
|---|---|---|---|---|---|
| | D5 Low VOC | D6 Low VOC | Example 15 | Example 16 | Example 17 |
| Potassium Oleate | 4.0 | 4.0 | 4.0 | 4.0 | 4.0 |
| Polysorbate 80 | 10.0 | 10.1 | 6.0 | 6.0 | 6.0 |
| d-Limonene | 10.1 | | 5.1 | 5.1 | 3.0 |
| Isopropyl Alcohol | | | 7.1 | 7.1 | 7.1 |
| Methyl Lactate | 5.1 | 31.4 | 13.1 | 5.0 | 13.1 |
| Water | | | 8.0 | 8.0 | 8.0 |
| Methyl Acetate | 26.3 | | 12.2 | 20.3 | 12.2 |
| Methyl 5-Methyl-2- | | | | | 2.1 |
| Furoate | | | | | |
| VOC | 21.72% | 17.83% | | | |

TABLE 4

| | Example 18 | |
|---|---|---|
| | Low VOC | Example 19 |
| Potassium Oleate | 4.0 | |
| Polysorbate 80 | 6.0 | |
| d-Limonene | 3.0 | 2.5 |
| Isopropyl Alcohol | 7.1 | |
| Methyl Lactate | 5.0 | 17.5 |
| Water | 8.0 | |
| Methyl Acetate | 20.3 | 27.5 |
| Methyl 5-Methyl-2- | 2.3 | 2.5 |
| Furoate | | |
| VOC | | |

### Methods for Removing Coatings on Various Materials

A. Stainless Steel Metal Plates.

Rustoleum® Black Semi Gloss protective enamel spray paint, Rustoleum® Red Gloss protective Enamel Spray paint, Perfomix Plastic Dip Rubber paint, and Valspar Interior/Exterior re spray paint for plastic were all applied to multiple clean stainless steel sheets (approximately 4×6 inches). The coating on each of the sheets was allowed to dry for a minimum of 24 hours. For one test the formulation in Example 9 (low VOC) and a second formulation with methyl 5-methyl-2-furoate in Example 18 were applied to the plates. Within approximately 5 to 8 minutes the paints on the various metal sheets bubbled up and the sheet could be wiped clean with a paper towel.

A nitrocellulose-based nail polish that was developed as a safer nail polish was painted on the metal sheets using 3 coats of the polish. Ten minutes was allowed between the painting of each coat. After the addition of the 3 coats the nail polish was cured for 24 hours. Multiple colors were tested: White, Brown, Black, Yellow, Orange, Blue, Red, Burgundy, and Pink. All 19 formulations were used to remove the nail polish by treating a cotton ball and wiping. All colors were removed with no smearing as is typical for nail polish removal. The same testing was performed on finger nails using the red polish. The formulations removed the nail polish without smearing or drying the nail as occurs with present commercial nail polish removers.

The following commercial nail polishes were tested as above using the 19 examples of formulations given in the preceding tables:

1. Sally Hansen Instant Dri® Re-Teal

2. RGB Nail Polish Orange

3. Sally Hansen Hard as Nails® Blue Boom Glitter

4. Mineral Fusion® Crimson Rouge

5. OPI Nail Lacquer Red

6. Sally Hansen Hard as Nails® Glitter Rock Star Pink

7. Gabriel Nail Polish Red

8. OPI Base/Gel Coat Chick Flick Cherry

9. Sally Hansen Insta Dri® Flashy Fuchsia

10. Sally Hansen Insta Dri® Cherry Fast.

Roof tar and super glue were applied to separate metal sheets and each was allowed to dry for 24 hours. Example 9 with low VOC was used to wipe the roof tar metal sheet and the super glue metal sheet. In both cases the stainless steel sheet was wiped clean.

An epoxy-nylon based coating was applied to metal sheets and allowed to dry for 24 hours. Example 9 (4.0 grams of potassium oleate, 6.0 grams of polysorbate 80 grams, 5.1 grams of D-Limonene, 7.1 grams of acetone, 5.1 grams of methyl lactate, 8 grams of water and 20.2 grams of methyl acetate with a measured VOC of 17.08%)

An epoxy-nylon based coating was applied to the stainless steel metal sheets and allowed to cure for 24 hours. Example 9 was applied to the treated sheet and allowed to penetrate the material for 10 minutes. After the 10 minutes the coating could be pulled off completely from the sheet.

B. Brick

Rustoleum® Black Semi Gloss protective enamel spray paint, Rustoleum® Red Gloss protective Enamel Spray paint, and Valspar Satin Interior pain Pink were applied separately to bricks. The paint was allowed to dry for 24 hours. Example 9 was applied to the coating on the bricks and sat for 10 minutes prior to removal. The 3 paint types were easily wiped off with a paper towel leaving no residual paint.

C. Iron Pipe

A piece of iron pipe was painted with Sally Hansen Miracle Gel™ Red nail polish on side and the other side was coated with a black Sharpie®. The coatings were dried for 24 hours. Example 9 and Example 18 (4.0 grams of potassium oleate, 6.0 grams of Polysorbate 80, 3.0 grams of D-Limonene, 7.1 grams of Isopropyl Alcohol, 5.0 grams of methyl lactate, 8.0 grams of water, 20.3 grams of methyl acetate and 2.3 grams of Methyl 5-Methyl-2- Furoate) were used to remove the polish. Neither acetone nor the example formulations removed the black Sharpie®.

D. Plastics

A PVC pipe, CPVC pipe and a PETE plastic bottle were painted with Sally Hansen Miracle Gel™ Red nail polish on one side with the other side was coated with a black Sharpie®. After 24 hours of drying, Examples 9 and 18 were used to remove the nail polish. Neither acetone nor the example formulations removed the black Sharpie®.

E. Silk Screen

A silk screen was coated with ink. Example 18 was utilized, and it removed entirely the ink and left the screens in a condition where no further cleaning was necessary.

Unexpectedly it was found that the various composition examples could be used to remove various coatings from a multitude of surfaces, both porous and nonporous.

While this invention has been described with respect to various specific examples and embodiments, it is to be understood that the invention is not limited thereto and that it can be variously practiced with the scope of the following claims. As used herein, the terms “comprises,” “comprising,” “includes,” “including.” “has,” “having, or any other variation thereof, are intended to cover a non-exclusive inclusion.

What is claimed is:

1. A composition for removing coatings from a wide variety of surfaces that is a mixture of compounds selected from sugar derived esters, a surfactant or surfactants, a co-solvent, and optionally a scent.

2. The composition of claim 1 wherein the sugar derived ester is selected from the group consisting of methyl lactate, methyl acetate and methyl-5-methyl-2-furoate.

3. The composition of claim 1 further wherein the surfactant is selected from the group consisting of potassium oleate, polysorbate 80, and PEG 7 glyceryl cocoate.

4. The composition of claim 1 further comprising about 4% to about 10% d-Limonene by weight.

5. The composition of claim 3 comprising between about 5% to about 15% by weight of surfactant or surfactants.

6. The composition of claiml comprising between about 0% to about 15% weight of surfactant or surfactants.

7. A coating removal composition of claim 1 having at least 30% or 35%, or at least 50% of a sugar derived ester by weight, about 0% to 10% by weight of methyl-5-methyl-2-furoate (Me408), at least 4%, or at least 6%, or at least 8%, or at least 10% by weight of d-limonene, at least 5%, at least 10%, or at least 15% by weight of a surfactant or surfactants with the remainder being a solvent.

8. A coating removal composition having at least 30% or 35%, or at least 50% of a sugar derived ester by weight, 5, about 0% to 10% by weight of methyl-5-methyl-2-furoate (Me408), at least 4%, or at least 6%, or at least 8%, or at least 10% by weight of d- limonene, at least 5%, at least 10%, or at least 15% by weight of a surfactant or surfactants with the remainder being a solvent.

9. A coating removal composition having at least 30% or 35%, or at least 40% of a first sugar derived ester by weight, about 5% or about 5% or about 10% of second sugar derived ester by weight, about 0% to 10% by weight of methyl-5-methyl-2-furoate (Me408), at least 4%, or at least 6%, or at least 8%, or at least 10% by weight of d- limonene, at least 5%, at least 10%, or at least 15% by weight of a surfactant or surfactants with the remainder being a solvent.

10. The coating removal composition of claim 7 wherein application of the composition for removing polish from a nail does not smear the polish on the nail.

11. A method of removing nail polish from a surface comprising applying an effective amount of the composition of claim 7 to the surface and removing the nail polish.

12. A method of removing ink from a silk screen comprising applying an effective amount of the composition of claim 7 to the surface of the silk screen.

13. A method of removing paint from a surface comprising applying an effective amount of the composition of claim 7 to the surface of the painted material.
